(19) United States
(12) Patent Application Publication
(10) Pub. No.: US 2017/0276671 A1
(43) Pub. Date: Sep. 28, 2017

(54) RECEPTOR LINKED REGENERATED CELLULOSE MEMBRANE AND METHODS FOR PRODUCING AND USING THE SAME

(71) Applicant: NB Postech, Inc., Pohang-si, Gyeongsangbuk-do (KR)

(72) Inventors: Joon Won PARK, Pohang-si, Gyeongsangbuk-do (KR); Sung Min SEO, Seoul (KR); Jong Myung KO, Pohang-si, Gyeongsangbuk-do (KR)

(73) Assignee: NB Postech, Inc., Pohang-si, Gyeongsangbuk-do (KR)

(21) Appl. No.: 15/504,647

(22) PCT Filed: Nov. 14, 2016
(86) PCT No.: PCT/KR2016/013105

§ 371 Date: Feb. 16, 2017

(30) Foreign Application Priority Data
Nov. 18, 2015 (KR) 10-2015-0161585

(51) Int. Cl. G01N 33/531; G01N 33/68; G01N 33/543

(52) U.S. Cl. CPC G01N 33/531; G01N 33/543; G01N 33/68; G01N 33/6803; G01N 33/6845; G01N 33/54353; G01N 33/00

(57) ABSTRACT

The present invention provides a regenerated cellulose membrane (RCM) that is useful in analysis of a sample for the presence of or the amount of a target molecule present in the sample. The invention also provides a method for producing and using the same. The RCM of the invention has a plurality of RCM functional groups on its surface in which a linker is covalently attached. The linker has a distal end and a proximal end, where the proximal end comprises at least one RCM linking functional group such that the RCM linking functional group is attached to the RCM functional group. The distal end of the linker comprises a receptor linking functional group that is used to covalently attach a receptor molecule. The receptor molecule is adapted for binding to a target molecule of interest when the target molecule is present in the sample. The RCM of the invention can be used for quantitative and/or qualitative analysis of a target molecule within the sample. The present invention also provides a method for producing and using the linker.

### TECHNICAL FIELD

The present invention relates to a regenerated cellulose membrane (RCM) having a linker that is adapted for attaching a receptor molecule for determining the presence of or the amount of a target molecule in a fluid sample. The present invention also relates to a method for using and producing such RCMs. In particular, methods of the invention can be used for quantitative or qualitative analysis of target molecules which selectively react with receptor (e.g., capture) molecules which are immobilized on the surface of a regenerated cellulose membrane via a linker

### BACKGROUND ART

A membrane is a porous material mainly made of a polymer such as mixed cellulose ester, Teflon®, nylon and the like. Typically, the diameter of a membrane pore ranges from 26 nm to 15 mm. Generally, membranes are used to remove non-desirable particles such as dust from a gas or a liquid. Membranes have also been used to sterilize solutions, often non-thermally. In addition, membranes have also been used to purify or concentrate according to a molecular weight (MW) of a protein or a nucleic acid such as DNA and RNA. Furthermore, membranes have been used to filter microorganisms such as bacteria or yeast.

Membranes are also used in diagnostics, such as in pregnancy test kits. For diagnostic use of membranes, typically a capture (i.e., receptor) molecule, such as an antibody or an enzyme, is immobilized on the membrane surface. A sample to be tested is applied or contacted with the membrane to allow binding of a desired target molecule, if present in the sample, to the capture (or receptor) molecule that is present on the surface of the membrane. Typically, the sample is allowed to flow into pores of the membrane via capillary force and travels laterally. This type of analysis is well known to one skilled in the art and are generally referred to as a lateral flow analysis.

Another method is to allow the fluid sample (gas or liquid) to flow through the membrane. Such methods are also well known to one skilled in the art and are known conventionally as a flow through analysis. See, for example, Ramachandran et al., “A Rapid, Multiplexed, High-Throughput Flow-Through Membrane Immunoassay: A convenient Alternative to ELISA,” Diagnostics, 2013, 3, 244-260; Le Goff et al., “Robust, High-Throughput Solution for Blood Group Genotyping,” Anal. Chem., 2010, 82, 6185-6192; and Le Goff et al., “Multipurpose high-throughput filtering microarrays (HiFi) for DNA and protein assays,” Biosensors and Bioelectronics, 2010, 26, 1142-1151.

A nitrocellulose membrane is a porous membrane (e.g., typically having 0.2 μm to 15 μm pore size) and electrostatically binds proteins through interaction between strong dipole of nitrate esters and strong dipole of a peptide bond and/or charges that may be present on the protein. The nitrocellulose membrane is overall electrically neutral and has no acidic hydronium ions. The absorption ability for the protein depends on the pH of an immobilization solution. The pH may influence immobilization efficiency of a specific protein by changing its characteristics in the solution. The nitrocellulose membrane can also electrostatically bind a nucleic acid that is negatively charged or has a separation of charges. The nitrocellulose membrane typically has a higher affinity for a single-stranded DNA and a DNA-RNA hybrid compared to double stranded DNA or an RNA. Thus, the nitrocellulose membrane is often used for separation or quantification of a single-stranded nucleic acid or separation or quantification of a nucleic acid forming a complex with a protein.

### DISCLOSURE OF INVENTION

### Technical Problem

However, analysis methods using a nitrocellulose membrane are based on electrostatic-based affinity, which is a noncovalent bond. Noncovalent bond include an ionic bond, a hydrogen bond, a hydrophobic interaction, and Van der Waals' interaction. Because nitrocellulose membrane uses noncovalent bond interaction to detect the presence of or the amount of the desired target molecule in a sample, it is difficult to reliably reproduce the results.

Accordingly, there is a need for a membrane based diagnostic method that does not utilize noncovalent bond attachment of a receptor (or capture) molecule.

### Solution to Problem

One aspect of the invention provides a membrane comprising a covalently attached receptor (or capture) molecule on its surface. The present invention also provides an analytical method for determining the presence of or the amount of a target molecule in a sample by covalently attaching a receptor (or capture) molecule on the surface of the membrane.

In some embodiments, the method includes adding a signal generating (or detection) molecule, whereby a signal is generated when a receptor molecule-target molecule binding pair is present on the membrane. Still in other embodiments, a signal generating molecule is combined with the target molecule, and the resulting complex is captured or bound to the receptor molecule.

The receptor molecule is selectively bound to the target molecule to form a receptor molecule-target molecule binding pair. Signal generated by the signal generating molecule is then analyzed to determine the presence of or the amount of target molecule in the fluid sample. In contrast to conventional nitrocellulose membrane diagnostic method, in some embodiments methods of the invention result in a highly reproducible signal according to a concentration of the target molecule, thereby allowing quantitative and/or qualitative analysis of the target molecule.

Yet in other embodiments, the distance between the receptor molecules covalently immobilized or bound to the membrane is controlled to prevent lateral steric hindrance and/or excessive absorption and/or loss of the receptor molecules immobilized on the membrane surface. Such an embodiment further increases reproducibility of the results.

One particular aspect of the invention provides a regenerated cellulose membrane (RCM) that is adapted for or useful in quantitative and/or qualitative analysis of a target molecule. In one embodiment, the receptor molecule is covalently attached to the surface of the RCM through a linker

Other aspects of the invention provide a method for using the same and a kit for quantitative and/or qualitative analyzing a target molecule comprising such an RCM.

Still other aspect of the invention provides a linker compound of the formula:

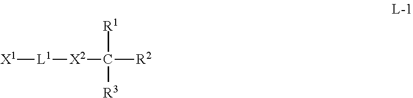

where X¹ is said receptor linking function group; X² is a branch linking functional group; L¹ is a first linker moiety having at least two, typically at least three and often at least four atoms in a chain; R¹ is a moiety of the formula -[L²-Q²]ₐ-{(L³-Q³)_{b}-[(L⁴-Q⁴)_{c}-(L⁵-Y)ₓ]_{y}}_{z}; each of R² and R³ is independently hydrogen, alkyl, or a moiety of the formula: -[L²-Q²]ₐ-{(L³-Q³)_{b}-[(L⁴-Q⁴)_{c}-(L⁵-Y)ₓ]_{y}}_{z} each of a, b, and c is independently 0 or 1; x is 1 when c is 0 or when c is 1, x is an integer from 1 to the oxidation state of Q⁴-1; y is 1 when b is 0 or when b is 1, y is an integer from 1 to the oxidation state of Q³-1; z is 1 when a is 0 or when a is 1, z is an integer from 1 to the oxidation state of Q²-1; each of Q², Q³ and Q⁴ is independently a branch atom having the oxidation state of at least 3; and each of L², L³, L⁴, and L⁵ is independently a linker moiety having at least two, typically at least three and often at least four atoms in a chain; provided the product of n, x, y, and z is at least 1.

### Advantageous Effects of Invention

The present invention provides a regenerated cellulose membrane (RCM) that is useful in analysis of a sample for the presence of or the amount of a target molecule present in the sample. The invention also provides a method for producing and using the same. The RCM of the invention has a plurality of RCM functional groups on its surface in which a linker is covalently attached. The linker has a distal end and a proximal end, where the proximal end comprises at least one RCM linking functional group such that the RCM linking functional group is attached to the RCM functional group. The distal end of the linker comprises a receptor linking functional group that is used to covalently attach a receptor molecule. The receptor molecule is adapted for binding to a target molecule of interest when the target molecule is present in the sample. The RCM of the invention can be used for quantitative and/or qualitative analysis of a target molecule within the sample. The present invention also provides a method for producing and using the linker.

### BRIEF DESCRIPTION OF DRAWINGS

FIG. 1 is a schematic diagram of a regenerated cellulose membrane (RCM) in which a linker is covalently attached to the surface of the RCM according to an exemplary embodiment of the present invention.

FIG. 2 is a schematic diagram of the RCM in which a capture molecule is attached to the linker

FIG. 3 is a schematic diagram of the RCM in which a target molecule is bound to the receptor (i.e., capture) molecule.

FIG. 4 is a schematic diagram of the RCM in which a detection (i.e., signal generating) molecule with a signal generating moiety is bounded to the receptor molecule-target molecule binding pair.

FIG. 5 is a graph of a fluorescent signal result using the RCM of the invention in a lateral flow type method according to an exemplary embodiment of the present invention.

FIG. 6 a graph of a fluorescent signal result using the RCM of the invention in a lateral flow type method according to another exemplary embodiment of the present invention.

FIG. 7 a graph of fluorescent signal result using the nitrocellulose membrane (NCM) in a lateral flow type method according to an exemplary embodiment of the present invention.

FIG. 8 shows colored and fluorescent signals of linkers having different number of anchoring functional group attached to the surface of the RCM according to an exemplary embodiment of the present invention.

### BEST MODE FOR CARRYING OUT THE INVENTION

Some aspects of the present invention provide a regenerated cellulose membrane (RCM) for quantitative or qualitative analysis of target molecules. Such RCM includes a linker that is covalently attached to its surface. The linker includes a proximal end and a distal end. In addition, the linker includes one or more branched moieties and a linear moiety in which one or more ends of the branched moieties are covalently attached to the functional group that is present in the RCM. The linker also includes a functional group that is capable of attaching a receptor (or capture) molecule. As used herein, the RCM refers to materials manufactured by the conversion of natural or synthetic cellulose to a soluble cellulosic derivative and subsequent regeneration, typically forming a fiber (via polymer spinning), a film (via polymer casting), a sheet or a filter.

Another aspect of the present invention provides a method for using the RCM to determine the presence of or the amount of a target molecule in a sample. Determination can be quantitative or qualitative. Such a method includes immobilizing, typically covalently, a capture or a receptor molecule at the end of a linear portion of the linker The sample is then contacted with a sample to determine whether a target molecule is present in the sample or the amount of target molecule present in the sample. The receptor is selected such that it selectively binds to the desired target molecule. As used herein, unless the context requires otherwise, the terms “receptor molecule” or simply “receptor”, “capture molecule,” “probe molecule” and “probe” are used interchangeably herein and refer to any molecule that can selectively bind to a desired target molecule. In addition, unless the context requires otherwise, the terms “ligand molecule” (or simply “ligand”) and “target molecule” (or simply “target”) are used interchangeably herein and refer to any molecule that can selectively bind to a receptor molecule.

It should be appreciated that the terms “ligand” and “receptor” do not refer to any particular substance or size relationship. These terms are only operational terms that indicate selective binding between the ligand or the target molecule and the corresponding receptor or capture molecule. In general, the moiety that is bound to the RCM surface is referred to as a receptor or capture molecule or a probe and any substance that selectively binds to the receptor molecule is referred to as a target molecule or a ligand. Thus, if an antibody is attached to the RCM surface then the antibody is a receptor molecule and the corresponding antigen is a target molecule. However, if an antigen is attached to the RCM surface then the antigen is a receptor molecule and the corresponding antibody is a target molecule.

Exemplary receptor molecules include, but are not limited to, antigens, antibodies, oligonucleotides (including DNAs, RNAs or fragments thereof), oligopeptides (including proteins, hormones, etc.), enzymes, substrates, drugs (e.g., a small organic molecule), drug-receptors, cell surface, receptor agonists, partial agonists, mixed agonists, antagonists, response-inducing or stimulus molecules, pheromones, transmitters, autacoids, growth factors, cytokines, prosthetic groups, coenzymes, cofactors, precursors, vitamins, toxins, regulatory factors, haptens, carbohydrates, molecular mimics, structural molecules, effector molecules, selectable molecules, biotin, digoxigenin, crossreactants, analogs, competitors or derivatives of these molecules as well as library-selected nonoligonucleotide molecules capable of specifically binding to selected targets and conjugates, and any other molecule that binds selectively with a receptor molecule.

The method can also include adding a signal molecule to the resulting mixture. The signal molecule includes a signal generating moiety such that when the signal molecule binds to the receptor molecule-target molecule binding pair, a signal is generated to allow detection the presence of the target molecule. The signal molecule can be a separate compound that is added to the mixture or alternatively, the target molecule can be linked to a signal generating moiety prior to binding to the receptor molecule.

Another aspect of the invention provides a kit for quantitatively or qualitatively detecting the presence of a target molecule. The kit includes a sample port for adding a sample to the sample pad. The kit also includes the RCM portion as described herein. The kit can also include a specific area where the receptor is covalently attached to the RCM (i.e., a “test area” or “test line”). In this manner, as the sample travels or flows from one end of the kit to the other end, the desired target molecule, if present in the sample, is captured by the receptor that is present in the test area. As a self-test for any errors, the kit can also include a control area or a control line. One method of using the control line is to attach a specific receptor molecule to the control line and adding a particular target molecule that binds to the receptor molecule on the control line. In this manner, if no signal is produced by on the control line, the results are discarded entirely as being erroneous.

Still another aspect of the invention provides a linker that includes one or more branched portion (or moieties) and a linear portion (or moiety) in which one or more ends of the branched moieties are attached to the surface of the RCM. The linker has a distal end and a proximal end. The proximal end of the linker comprises at least one RCM linking functional group such that the RCM linking functional group is attached to the RCM functional group (i.e., a functional group that is present on the surface of the RCM). The distal end of the linker comprises a receptor linking functional group. This receptor linking functional group is used to attached a functional group of a receptor molecule.

Still yet another aspect of the invention provides a method of preparing a linker

The RCM of the invention and the methods disclosed herein provide numerous advantages of detecting a target molecule in a sample. One particular advantage of the RCM of the invention includes production of signals having high reproducibility. Without being bound by any theory, it is believed this is due at least in part to covalent immobilization of the capture molecule using the linker to the RCM. In addition, it is believed it is also due to a relatively uniform distribution of the capture molecule. Furthermore, use of the linker disclosed herein is believed to results in a regular interval spacing of the capture molecule, thereby allowing a better binding between the capture molecule and the target molecule. It is also believed that regular interval spacing prevents abnormal signal generation (i.e., signals having much larger or smaller values than the expected value) due at least in part by avoiding excessive binding or undesirable loss of the capture molecule, which are frequently observed when a noncovalently attached capture molecules are used.

The modified regenerated cellulose membrane (or simply “regenerated cellulose membrane” or “RCM”) of the invention can be used for quantitative and/or qualitative analysis of target molecules. In one particular embodiment, the RCM includes a linker comprising one or more branched portion or moieties and a linear portion or moiety in which one or more ends of the branched moieties comprises an RCM linking functional group that are used to covalently linked to a functional group that is present on the surface of the RCM, such as hydroxyl group (—OH), an amino group (—NH₂), a thiol group (—SH) etc. The distal end of the linker includes a receptor linking functional group that can be utilized to covalently attach a receptor molecule. As used herein the term “covalent” when referring to linkage or bond means a molecular bond or a chemical bond that involves the sharing of electron pairs between atoms. The terms “non-covalent bond” and “noncovalent interaction” are used interchangeably herein and refer to interactions such as, but are not limited to, electrostatic interactions (e.g., ionic bonding, hydrogen bonding and halogen bonding), Van der Waals forces, ?-effects, hydrophobic effect or interaction, etc.

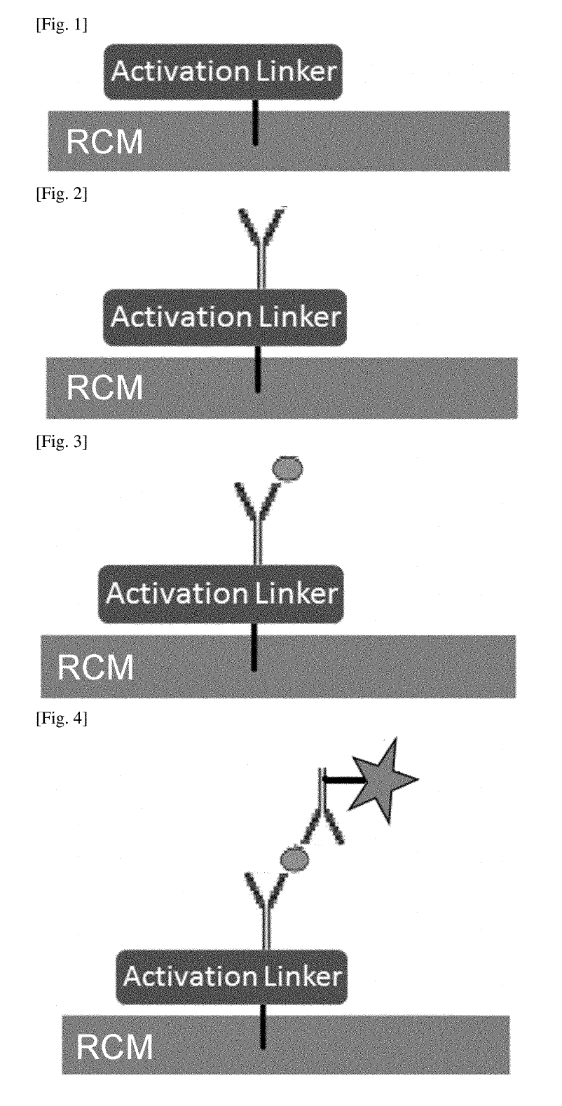

In one particular embodiment, the RCM linking function group of the linker is covalently attached to a hydroxyl group that is present in the surface of RCM. Suitable RCM for attaching a linker are commercially available (e.g., RC 60, pore size 1 mm, thickness 90 mm, GE healthcare). In general any commercially available RCMs can be used. In another embodiment, the linker includes a plurality of RCM linking functional groups, i.e., a linker having a plurality of branched ends. The distance between the RCM linking functional groups and a density of the resulting linkers on the RCM are typically determined by the size and/or the number of branched ends (see FIG. 1).

A receptor molecule that can bind selectively to a desired target molecule is typically attached to the linker in the following manner. A solution of receptor molecule is contacted with the RCM having a linker bound to its surface. This allows covalent attachment of the receptor molecule to the linker (see FIG. 2). As used herein, when describing a chemical reaction the term “treating”, “contacting” or “reacting” refers to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The method of the invention includes contacting a sample to be tested for the presence of or the amount of the target molecule with the RCM having a capture molecule covalently attached to its surface. As discussed above, the capture molecule is capable of selectively binding to the desired target molecule whose analysis is desired. When the target molecule is present in the sample, it binds to the capture molecule. See FIG. 3. The analysis can be a lateral flow process, a flow through process or any other process known to one skilled in the art.

In some embodiments, a detection (or a signal generating) molecule having a signal generating moiety such as a fluorescent molecule, colloidal gold, or HRP is contacted with the resulting RCM. Alternatively, the target molecule can be tagged or labeled with the signal generating molecule prior to binding with the capture molecule. If the target molecule is present in the sample, it will bind to the receptor molecule to produce a receptor molecule-target molecule binding pair. Regardless of when the signal generating molecule is added, the formation of this receptor molecule-target molecule binding pair can be detected by observing the signal generated by the signal generating moiety. See FIG. 4. As expected, depending on the signal generating moiety, different signals are generated. For example, when the signal generating moiety is a fluorescent molecule, it generates a fluorescent light. When the signal generating moiety is colloidal gold, it can be discernible by naked eyes or using an optical equipment. And if the signal generating moiety is an enzyme such as HRP, it generates an amplified signal.

The present invention also provides a method for highly reliable quantitative or qualitative analysis of bio-target molecules such as proteins, DNAs, RNAs, PNAs, aptamers, ligands, lipids, inorganics, and cells using the RCM. The term “about” when referring to a numeric value means ±20%, typically ±10%, often ±5%, and more often ±2% of the numeric value. The term “highly reliable” means the standard deviation of the assay performed at least 3 times using the same condition is about ±15% or less, typically about ±10% or less, and often about ±5% or less of the mean.

The method of the invention includes covalently attaching an RCM linking functional group on the proximal end of the linker to the functional group that is present on the surface of a regenerated cellulose membrane; covalently attaching a receptor linking functional group that is present in the distal end of the linker to a capture molecule; treating a sample to be tested with a detection (or signal generating) molecule to form a target molecule-signal generating molecule complex, if the target molecule is present in the sample; contacting the resulting sample to the RCM having a covalently attached capture molecule; and detecting and/or quantifying the signal generated by the signal generating molecule. It should be appreciated that in this embodiment the signal is generated only if a triple complex of the capture molecule-target molecule-signal generating molecule is present on the surface of the RCM. It should also be appreciated that the signal-generating molecule can be added to the RCM after contacting a sample (without the signal generating molecule) to the RCM having covalently attached capture molecule.

If the receptor linking functional group is protected, the method of the invention can also include activating (or deprotecting) the receptor linking functional group of the linker prior to or simultaneously (e.g., in situ deprotection) with contacting the receptor molecule with the RCM having the linker attached to its surface.

The sample for analysis can be any fluid sample (e.g., gas or liquid) that may or may not include the target molecule. Exemplary samples include, without limitation, biological samples such as blood, plasma, serum, saliva, urine, feces, sputum, and tissue; environmental samples such as water samples, soil samples; air samples; food samples such as meat, fish, vegetables, beverages, dried products; and processed products.

Exemplary target molecules that can be analyzed by the methods of the invention include, but are not limited to, any material that requires analysis, such as antigens, antibodies, proteins, peptides, DNAs or a fragment thereof, RNAs or a fragment thereof, PNAs, aptamers, ligands, toxic compounds, lipids, hormones, inorganic compounds, bacteria, viruses, macro-vesicles or micro-vesicles, and cells. In general, the capture molecule is any molecule that selectively binds to the desired target molecules.

The signal generating molecule of includes a signal generating moiety such as, but not limited to, fluorophores, phosphors, magnetic particles, nanoparticles, nanofibers, and the like.

In the present invention, the distance between the receptor linking functional groups of the linker and the density of linkers that are bound to the RCM can be adjusted according to the size and the structure of the linker. In some embodiments, the distance between the receptor linking functional groups on the linkers that are covalently attached to the RCM ranges from about 0.5 nm to about 20 nm. In other embodiments, the density of the linkers bound to the RCM surface ranges from about 0.001 molecule/nm² to about 4 molecule/nm².

The present invention also provides a kit comprising the RCM of the present invention. Such a kit can be used for quantitative and/or qualitative analysis of the target molecules, for example, in analyzing for diseases etc. Typically, the kit also includes a receptor molecule already attached to the RCM or it can be provided as a separate component so that it can be attached prior to analyzing a sample. Depending on the disease to be analyzed or a desired target molecule to be analyzed, the receptor molecule can be separately prepared or purchased by the user. The kit can be made to be useful for a lateral through type or a flow through type analysis. However, it should be appreciated that the scope of the invention is not limited to such types of analysis. In general, any analytical methods that are known to one skilled in the art can be used as long as they are suitable or adaptable for using the RCM of the present invention.

Other aspects of the invention provide a linker and a method for preparing the same. In one particular embodiment, the linker in its simplest form is a compound of the formula: Z^{a}-Y^{a}-Q^{a}-[Z^{b}]ₙ₋₁, where Z^{a} is a linear portion (or a linear moiety or the “distal end of the linker”) in which a receptor linking functional group is present. The branched portion (or the “proximal end of the linker”) comprises -Q^{a}-[Z^{b}]ₙ₋₁. Y^{a} is a functional group that links the linear portion to the branched portion of the linker. In some cases, Y^{a} is absent, i.e., the linear portion and the branched portion are linked directly without any other functional group, for example, when Q^{a} is —OP—[Z^{b}]ₙ₋₁ or —OS—[Z^{b}]ₙ₋₁ and each Z^{b} is independently alkyl, —OZ^{c}, ═O, etc. Q^{a} is an atom having oxidation state of at least 3, such as N, C, Si, P, S, etc. Typically Q^{a} is carbon. The variable n is the oxidation state of Q^{a}, for example, when Q^{a} is N, n is 3, when Q^{a} is C or Si, n is 4, when Q^{a} is P or S, n can be 3, 4, 5 or 6, etc. Each of Z^{b} is independently hydrogen, alkyl or a moiety of the formula: -[L²-Q²]ₐ-{(L³-Q³)_{b}-[(L⁴-Q⁴)_{c}-(L⁵-Y)ₓ]_{y}}_{z}, where each of a, b, and c is independently 0 or 1; x is 1 when c is 0 or when c is 1, x is an integer from 1 to the oxidation state of Q⁴-1; y is 1 when b is 0 or when b is 1, y is an integer from 1 to the oxidation state of Q³-1; z is 1 when a is 0 or when a is 1, z is an integer from 1 to the oxidation state of Q²-1; each of Q², Q³ and Q⁴ is independently a branch atom having the oxidation state of at least 3; and each of L², L³, L⁴, and L⁵ is independently a linker moiety having at least two, typically at least three, and often at least four atoms in a chain; provided the product of x, y, and z is at least 1, and also provided at least one of Z^{b} is a moiety of the formula: -[L²-Q²]ₐ-{(L³-Q³)_{b}-[(L⁴-Q⁴)_{c}-(L⁵-Y)ₓ]_{y}}_{z}. It should be appreciated that when only one of the Z^{b} moiety is a moiety of the formula: -[L²-Q²]ₐ-{(L³-Q³)_{b}-[(L⁴-Q⁴)_{c}-(L⁵-Y)ₓ]_{y}}_{z}, then it is possible that the branched portion -Q^{a}-[Z^{b}]ₙ₋₁ can actually be a none branched moiety when the product of x, y and z is 1. However, for the sake of brevity, even if only one of the Z^{b} moiety is a moiety of the formula: -[L²-Q²]ₐ-{(L³-Q³)_{b}-[(L⁴-Q⁴)_{c}-(L⁵-Y)ₓ]_{y}}_{z}, it will be called a “branched portion” throughout this disclosure.

The linker of the present invention includes one or more, typically at least two, and often at least three branched moieties and a linear moiety. The synthesis of the linker can be achieved by linking a functional group that is present on the branched portion with a functional group that is present on the linear portion. That is, the functional group Y^{a} is typically formed from a functional group that is present on the linear portion (Z^{a}) with a functional group that is present on the branched portion (-Q^{a}-[Z^{b}]ₙ₋₁). This can be illustrated by the following reaction:

Z^{a}-X^{a}+X^{b}-Q^{a}-[Z^{b}]ₙ₋₁→Z^{a}-Y^{a}-Q^{a}-[Z^{b}]ₙ₋₁

where the reaction between the functional group X^{a} and X^{b} results in a new functional group Y^{a}. For example, if one of X^{a} or X^{b} is a hydroxyl group and the other is an ester or a carboxylic acid, then the resulting Y^{a} is an ester linkage; when one of X^{a} or X^{b} is an amino group and the other is an ester or a carboxylic acid, then the resulting Y^{a} is an amide linkage; when one of X^{a} or X^{b} is a hydroxyl group and the other is a leaving group (e.g., halide, mesylate, tosylate, etc.), then the resulting Y^{a} is an ether linkage; when one of X^{a} or X^{b} is a thiol group and the other is an ester or a carboxylic acid, then the resulting Y^{a} is a thioester linkage, etc. In some embodiment, the final linker is produced by chemically forming a heteroaryl (e.g., triazole, imidazole, etc.) or a cyclic compound within the linear portion of the linker

As discussed herein, one or more ends of the branched portion of the linker include an RCM linking functional group that is used to covalently attach to the functional group that is present in the RCM surface. In one particular embodiment, the functional group that is present on the surface of the RCM is a hydroxyl group (—OH) and the RCM linking functional group that is present in the linker is a carboxylic ester.

The distal end of the linker includes a receptor linking functional group. This receptor linking functional group is used to covalently attach a receptor molecule for a desired target molecule. Exemplary receptor linking functional groups include, but are not limited to, a hydroxyl group, a formyl group, a carbonyl group, a carboxylic group, an ether group, an ester group, a nitro group, an amino group, a sulfonic acid group, a phenyl group, an alkyl group, a phosphine group, an N-hydroxysuccinimide (NHS)-ester group, an aldehyde group, an epoxide group, an lactone group, a carbonyl diimidazole group, a maleimide group, an iodoacetyl group, a pyridyl disulfide group, a hydrazide group, and 1-ethyl-3-(3-dimethylaminopropyl)carbodi-imide)?HCl (EDC). In one particular embodiment, the receptor linking functional group is selected from the group consisting of an aldehyde group and an epoxide group.

Exemplary RCM linking functional groups include, but are not limited to, a hydroxyl group, a formyl group, a carbonyl group, a carboxylic group, an ether group, an ester group, a nitro group, an amino group, a sulfonic acid group, a phenyl group, an alkyl group, a phosphine group, an aldehyde group, and an epoxide group, as well as other functional groups known to one skilled in the art that can be used to covalently attach the functional group on that is present on the surface of RCM, such as a hydroxyl group, an amino group or a thiol group.

The number of branched moieties of the linker (i.e., the product of x, y, and z) can range from 1 to 100, typically from 1 to 50, often 1 to 27, more often 1 to 9 and most often 3.

In one particular embodiment, the linker is a compound of the formula:

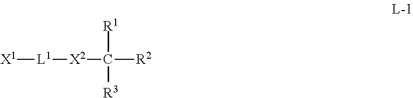

where X¹ is a receptor linking function group; X² is a branch linking functional group; L¹ is a first linker moiety having at least two, typically at least three and often at least four atoms in a chain. In some embodiment, L¹ is a moiety of the formula —R^{w}—Ar¹—R^{y}—, where each of R^{w} and R^{y} is independently alkylene, heteroalkylene (such —[(CH₂)ᵣ—X]ₛ—(CH₂)ₜ—,where each of r, s and t is independently an integer of 1 to 5, typically 2 to 4, and often 2 or 3 and X is O, NR or S where R is hydrogen, alkyl or a protecting group and Ar¹ is heteroaryl such as, but not limited to triazole, imidazole, etc.; R¹ is a moiety of the formula -[L²-Q²]ₐ-{(L³-Q³)_{b}-[(L⁴-Q⁴)_{c}-(L⁵-Y)ₓ]_{y}}_{z}; each of R² and R³ is independently hydrogen, alkyl, or a moiety of the formula: -[L²-Q²]ₐ-{(L³-Q³)_{b}-[(L⁴-Q⁴)_{c}-(L⁵-Y)ₓ]_{y}}_{z}; each of a, b, and c is independently 0 or 1; x is 1 when c is 0 or when c is 1, x is an integer from 1 to the oxidation state of Q⁴-1; y is 1 when b is 0 or when b is 1, y is an integer from 1 to the oxidation state of Q³-1; z is 1 when a is 0 or when a is 1, z is an integer from 1 to the oxidation state of Q²-1; each of Q², Q³ and Q⁴ is independently a branch atom having the oxidation state of at least 3; and each of L², L³, L⁴, and L⁵ is independently a linker moiety having at least two, typically at least three, and often at least four atoms in a chain; provided the product of n, x, y, and z is at least 1. Some specific linkers of the invention include, but are not limited to, 3,3′-((2-((2-carboxyethoxy)methyl)-2-(3-(1-(4-oxobutyl)-1H-1,2,3-triazol-5-yl)propanamido)propane-1,3-diyl)bis(oxy))dipropionic acid; 3,3′-((2-((2-carboxyethoxy)-methyl)-2-(3-(1-(2-(2-(2-oxoethoxy)ethoxy)ethyl)-1H-1,2,3-triazol-5-yl)propanamido)propane-1,3-diyl)bis(oxy))dipropionic acid; 3,3′-((2-((2-carboxyethoxy)methyl)-2-(3-(1-(2-(2-(2-(oxiran-2-ylmethoxy)ethoxy)ethoxy)ethyl)-1H-1,2,3-triazol-5-yl)propan-amido)propane-1,3-diyl)bis-(oxy))dipropionic acid; and 3,3′-(2-((2-carboxyethoxy)methyl)-2-(3-(1-(3-(oxiran-2-yl)propyl)-1H-1,2,3-triazol-5-yl)propanamido)propane-1,3-diyl)bis(oxy))di-propionic acid.

In some embodiments, each of L¹, L², L³, L⁴ and L⁵ is independently ethylene, propylene or a higher alkylene multiples thereof, e.g., —(CH₂)ₙ—, where n is an integer of 2 to 20, typically 2 to 10, and often 2 to 6, or an alkylene having a heteroatom inter-dispersed therein, e.g., —[X(CH₂)ₙ]ₘ—, where n is as defined above and m is an integer from 1 to 20, typically 1 to 10, often 1 to 5 and more often 1 to 3; X is —O—, —NR^{z}—, —S(O)ₖ—, —OS(O)₂O—, where R^{z} is hydrogen, alkyl, or a nitrogen protection group, and k is 0, 1 or 2.

Still in other embodiments, L¹ first linker moiety having at least four, typically at least six and often at least eight atoms in the chain. Yet in other embodiments, L¹ comprises a heteroaryl group such as triazole and/or imidazole.

Some of the exemplary methods for producing linkers of the invention are illustrated in Schemes IA-IE below:

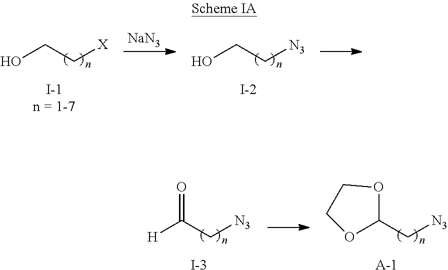

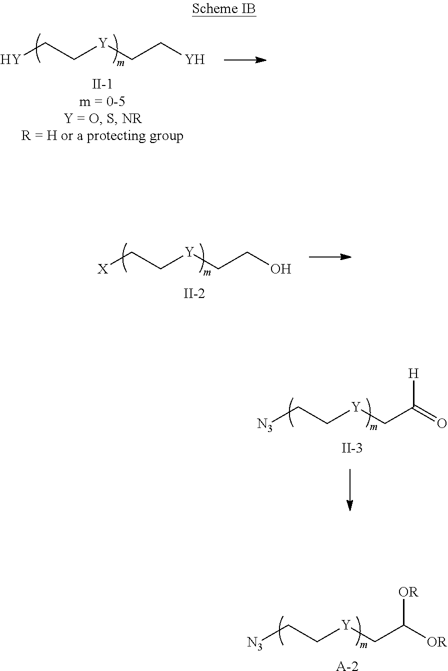

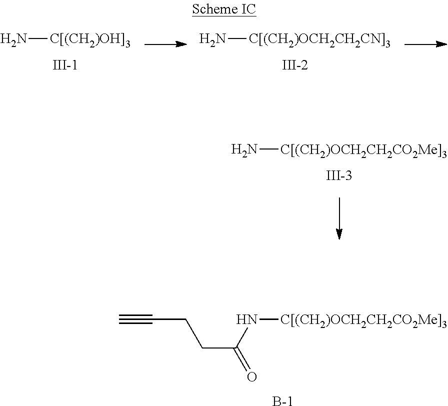

Referring to Scheme IA, a hydroxyl compound having a leaving group X (e.g., mesylate, tosylate, halide, etc.), I-1, is reacted with an azide compound to produce an azido alcohol I-2. The hydroxyl group is then oxidized to produce an aldehyde compound I-3, and the protection of the aldehyde functional group as an acetal then provides a protected compound A-1. As can be seen in Scheme IA, by using carbon chain of various length, a wide variety of compound A-1 is produced.

Referring to Scheme IB, an analog of compound A-1 is prepared in which one or more of the carbon atoms in the chain is replaced with a heteroatom such as O, S, or NR, where R is hydrogen, alkyl or a protecting group. By converting one of the Y group to a leaving group X and replacing the leaving group with an azide group then provides an azido compound. The other YH group (when Y is NR or S) is then converted to the hydroxyl group and the oxidation then provides an azido aldehyde compound II-3. Protection of the aldehyde group as an acetal then provides a protected azido compound A-2. As can be seen, Schemes IA and IB illustrate two particular embodiments for the synthesis of at least a portion of the linear portion of the linker

Referring to Scheme IC, a portion of the linker containing the branched portion is illustrated. In this process, Michael addition reaction of tris(hydroxymethyl)aminomethane, III-1, with acrylonitrile provides the compound III-2. The cyano groups are then converted to esters to produce the compound III-3. Coupling the amine group with 4-pentynoic acid then produced the compound B-1. It should be appreciated that using other alkynoic acid (e.g., propiolic acid, 3-butynoic acid, 5-hexynoic acid, 6-heptynoic acid, etc.) provides different chain length amide analogs of B-1. As shown in Scheme ID below, in some embodiments, by reacting the compound III-3 with tris(hydroxyl-methyl)aminomethane and repeating the processes allows branched portion of the linker with upto 100, typically up to 81 RCM linking groups. See, for example, compound III-4. Alternatively, the carboxylic acid groups of compound III-3 can be reduced first to an alcohol and can be reacted with other di-carboxylic acid compounds to produce other variations of the linkers. See compound III-5. In addition, the alcohol moiety of reduced compound of III-3 can be converted to an amino group and reacted with other dicarboxylic acid compounds to produce an amide linked compounds. See, for example, compound III-6.

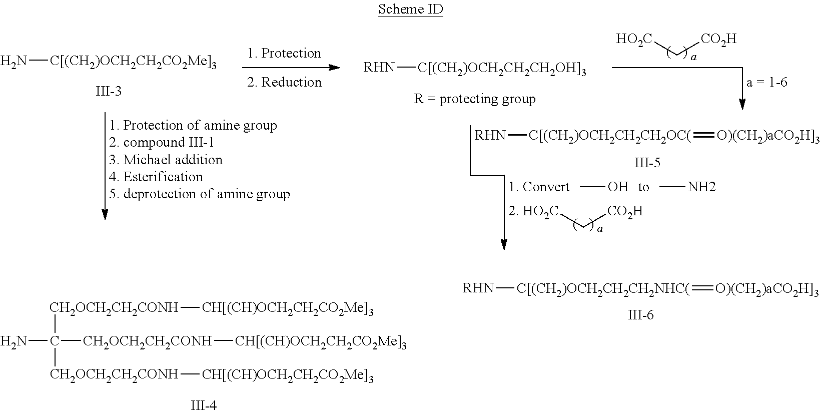

As shown in Scheme IE below, two portions (e.g., A-1 or A-2 with B-1) are then coupled using Click chemistry to produce a corresponding triazole compounds C-1 and C-2. The methyl ester is then saponified and acidified to produce the corresponding carboxylic acids, which are used as linkers. The carboxylic acid portions are used to covalently attach to the RCM and the protected aldehyde is deprotected and used as a receptor linking functional group to attach a receptor molecule. Alternatively, the aldehyde group can be converted to other functional groups such as an epoxide, a carboxylic acid, amine, a hydroxyl group, a thiol group, etc. which then can be used to attach a desired receptor molecule.

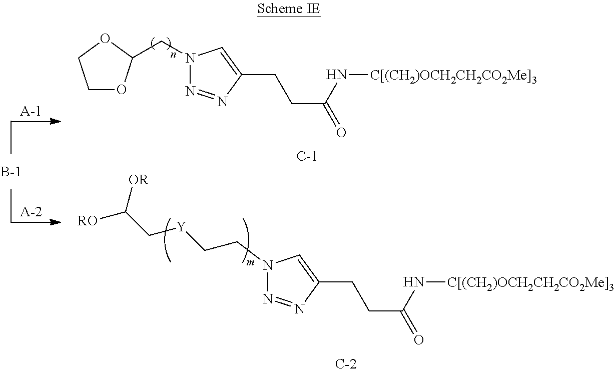

In this manner, a wide variety of linkers are produced using the methods described herein as well as in the Examples section.

The terms used herein are well known to one skilled in the art of organic chemistry, biochemistry, biology, and other life sciences. Some of the specific definitions of the terms used herein include the following definitions. The term “sample” refers to a fluid sample, e.g., gas or liquid including a solution, an emulsion, a dispersion, a suspension, and the like. The term “biological fluid” refers to all clinical samples such as blood, plasma, serum, urine or saliva, sweat, mucus, and includes all biological fluids that are internally or externally excreted, secreted or transferred by an organism. The term “aptamer” (or nucleic acid antibody) is used herein to refer to a single- or double-stranded DNA or a single-stranded RNA molecule that recognizes and binds to a desired target molecule by virtue of its shape. See, e.g., PCT Publication Nos. WO92/14843, WO91/19813, and WO92/05285, the disclosures of which are incorporated by reference herein.

The term “alkyl” refers to a saturated linear monovalent hydrocarbon moiety of one to twelve, preferably one to six, carbon atoms or a saturated branched monovalent hydrocarbon moiety of three to twelve, preferably three to six, carbon atoms. Exemplary alkyl group include, but are not limited to, methyl, ethyl, n-propyl, 2-propyl, tert-butyl, pentyl, and the like. “Alkylene” refers to a saturated linear saturated divalent hydrocarbon moiety of one to twelve, preferably one to six, carbon atoms or a branched saturated divalent hydrocarbon moiety of three to twelve, preferably three to six, carbon atoms. Exemplary alkylene groups include, but are not limited to, methylene, ethylene, propylene, butylene, pentylene, and the like.

“Protecting group” refers to a moiety, except alkyl groups, that when attached to a reactive group in a molecule masks, reduces or prevents that reactivity. Examples of protecting groups can be found in T.W. Greene and P.G.M. Wuts, Protective Groups in Organic Synthesis, 3rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxy-carbonyl, 9-fluorenylmethyloxycarbonyl (FMOC), nitro-veratryloxycarbonyl (NVOC), and the like.

The term “heteroaryl” means a monocyclic or bicyclic aromatic moiety of 5 to 12 ring atoms containing one, two, or three ring heteroatoms selected from N, O, or S, the remaining ring atoms being C. The heteroaryl ring can optionally be substituted with one or more substituents. Exemplary heteroaryl includes, but is not limited to, pyridyl, furanyl, thiophenyl, thiazolyl, isothiazolyl, triazolyl, imidazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

The antibody includes all of polyclonal antibodies purified from an antiserum obtained by immunizing a mammal with a particular antigen as well as a monoclonal antibody produced from a hybridoma. The antibody serving as the detection molecule marked with the signal entity can be a monoclonal antibody in order to obtain high selectivity for the target molecules, but when the antibody serving as the capture molecule recognizing the target molecules immobilized in the matrix is the monoclonal antibody, the antibody serving as the detection molecule marked with the signal entity may also be the polyclonal antibody. When referring to formation of a complex, the term “incubation” in the present invention is to be left at a predetermined temperature during the formation of a receptor molecule-target molecule binding pair formation (e.g., reaction between antigens and antibodies, or complementary DNAs and RNAs). The antigen-antibody reaction means specific-binding of the antigen to the antibody, and the reaction between the complementary DNAs and RNAs means that DNAs or RNAs of respective strands having complementary or near complementary base sequences (e.g., at least 90%, typically at least 95%, often at least 98%, and most often 100%) that can be hybridized through the reaction.

A reaction temperature in the incubation and the process of generating the signal of the signal entity in the present invention is not particularly limited, unless the reaction solution is not frozen or evaporated. Further, for use of an enzyme for a signal entity, the reaction temperature may be in a range of 4° C. to 100° C. so that its activity is not lost, but in the case of rapidly finishing the reaction by activating the enzymatic activity, the reaction temperature may be in a range of 15° C. to 100° C.

In the incubation process in the present invention, as the incubation time is increased, a reaction efficiency between the target molecule and the detection molecule and between the complex and the capture molecule is increased, and as a result, the intensity of the signal (fluorescence, coloring, luminescence, magnetism, and the like) is increased, but the binding reaction is saturated after a certain period. Further, in order to rapidly complete the measurement, it is required to shorten the incubation time. As the amount of the capture molecule supported on the RCM matrix is increased, more target molecules may be captured, and thus sensitivity is improved and the reaction time may also be shortened. On the contrary, if the amount is too large to free a binding epitope that captures the target molecules, the capturing efficiency may be deteriorated. Therefore it is desirable to optimize the amount of the capture molecule. In the present invention, since a distance between the target molecules immobilized on the RCM is controlled by selecting size of a linker, problems associated with conventional cellulose based methods (e.g., noncovalently attached receptor molecules, use of NCM, etc.) are overcome.

Further, in the present invention, the ‘fluorescence’, ‘chemiluminescence’, and ‘coloring’ are to show desired light (fluorescence or luminescence) signal or visible color by irradiating light or adding a reagent. Among the measuring methods of the present invention, the ‘coloring’ may verify the signal intensity with the naked eye semi-quantitatively or qualitatively, but use of an instrument can make the analysis quantitative. In addition, in the ‘chemiluminescence’ and ‘fluorescence’, intensity of the light can be measured with an image sensor and the sensor makes the analysis quantitative.

In the present invention, the ‘detection’ is to verify the presence of the target molecules and includes quantitative or semi-quantitative analysis of the concentration of the target molecules or qualitative analysis of verifying whether the target molecules are present. In a sample the target molecules and a complex formed from the target molecules and a detection molecules tagged with a signal entity are included and water and additives to facilitate the binding reaction with the capture molecules can be added, but not necessarily the sample is liquids. For example, when the sample is a solid, the sample may be dissolved by an appropriate solvent or dispersed in a solvent. Further, the sample amount required for measurement is not particularly limited so long as the amount is good enough to allow the mutual contact for the lateral flow method or the flow through method. Since extracted sample from blood or the like is limited, in many cases, the sample volume is small, and while the sample volume increased by the dilution, but the concentration of the target molecules is decreased by the process. As a result, detecting or quantify the target molecule often is challenging. To counteract the low concentration, frequently the extended incubation time is preferred. Sometimes, increased amount of the capture molecule and the detection molecule is a remedy. Typically, the preferred sample volume ranges from 10 μl and 200 μl.

In the present invention, the method of covalently immobilizing the capture molecule to the linker of the RCM matrix means covalent binding using functional groups, such as a hydroxyl group, a formyl group, a carbonyl group, a carboxylic group, an ether group, an ester group, a nitro group, an amino group, a sulfonic acid group, a phenyl group, an alkyl group, a phosphine group, an N-hydroxysuccinimide (NHS)-ester group, an aldehyde group, an epoxide group, an lactone group, a carbonyl diimidazole group, a maleimide group, an iodoacetyl group, a pyridyl disulfide group, a hydrazide group, and 1-ethyl-3-(3-dimethylaminopropyl)carbodi-imide).HCl (EDC), which are present on the end of the linear moiety of the linker attached to the RCM matrix. Treatment to reduce nonspecific adsorption of the target molecule and the complex is frequently performed to increase the signal-to-noise ratio. For example, a method called ‘blocking’ in which reagents of choice pre-occupy uncontrolled reactive sites of the RCM. When the target molecule is a protein, use of casein or bovine serum albumin is frequently adopted.

The target molecule in the present invention is not particularly limited, and includes a biomaterial such as an antigen, an antibody, a protein, a lipid, a peptide, a DNA, an RNA, a toxic compound, a mineral, a bacterium, a virus, a macro-vesicle, and a micro-vesicle, a non-biomaterial such as a drug, a pigment, heavy metal and a mineral, or the like. In the case of using antibodies as the capture molecule and the detection molecule, it is preferred that the target molecule has a molecular weight more than that of 12 to 15 amino acids. However, in the case of target molecule of a low molecular weight such as vitamin or glucose, competition assay can be overcome the difficulty Further, the target molecule in the present invention may include cytokines derived from various animals such as humans, mice, rabbits, pigs, cytokines produced by cultured cells, or the like as the origin of the cytokines, but are not limited thereto.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### MODE FOR THE INVENTION

### Example 1

### Synthesis of 2-(3-azidopropyl)-1,3-dioxolane

To a round bottom flask was added 4-chloro-1-butanol (5.0 g), NaN₃ (5.99 g) and DMF (30 mL). The mixture was stirred for 15 hrs under reflux. The resulting mixture was filtered through a Celite-pad. The filtrate was concentrated in vacuo, and the resulting residue was dissolved in 30 mL of ethyl acetate. The solution was washed with 20 mL of aqueous 1 N HCl, 20 mL of a saturated aqueous NaHCO₃ solution and 20 mL of a saturated NaCl solution. The organic layer was dried over anhydrous MgSO₄, filtered, concentrated and purified by column chromatography to yield 4-azido-l-butanol (yield=85%, colorless liquid). ¹H NMR (300 MHz, CDCl₃, ppm) 3.69 (t, J=5.82 Hz, 2H), 3.35-3.31 (m, 2H), 1.74-1.63 (m, 4H), 1.43 (bs, 1H).

To a round bottom flask was added 2-iodoxybenzoic acid (“IBX”, 4.833 g), 10 mL of DMSO, and the mixture was stirred at room temperate. To this solution was added 4-azido-l-butanol (1.656 g). The mixture was stirred for 5 hrs and quenched by adding water (30 mL). White solids were filtered and the aqueous layer was extracted with ethyl acetate (30 mL×3). The combined organic layer was washed with a saturated aqueous NaHCO₃ solution, a saturated aqueous Na₂CO₃ solution, water, and a saturated aqueous NaCl solution. The organic layer was dried over MgSO₄, filtered and concentrated to yield 4-azido-l-butanal. ¹H NMR (300 MHz, CDCl₃, ppm) 9.81 (s, 1H), 3.36 (t, J=6.60 Hz, 2H), 2.62-2.56 (m, 2H), 1.96-1.87 (m, 2H).

To a round bottom flask was added 4-azido-l-butanal (0.684 g), 10 mL of benzene (10 mL), ethylene glycol (2.74 mL) and p-toluenesulfonic acid (10.4 mg). The resulting mixture was heated to reflux for 12 hours. The solution was cooled to room temperature and saturated aqueous NaHCO₃ was added. The aqueous layer was extracted with ethyl acetate. The combined organic layer was washed with brine, dried over MgSO₄, filtered, concentrated and purified via column chromatography to yield 2-(3-azidopropyl)-1,3-dioxolane (“Compound A”). ¹H NMR (300 MHz, CDCl₃, ppm) 4.89 (t, J =1.54 Hz, 1H), 4.00-3.95 (m, 2H), 3.88-3.83 (m, 2H), 3.34 (t, J =1.54 Hz, 2H), 1.78-1.69 (m, 4H).

### Example 2

### Synthesis of dimethyl 3,3′-((2-((3-methoxy-3-oxopropoxy)methyl)-2-(pent-4-ynamido)propane-1,3-diyl)bis(oxy))dipropionate

To a round bottom flask was added tris(hydroxymethyl)aminomethane (7.5 g) and 1,4-dioxane (15 mL). The solution was cooled to 0° C. and KOH (40 wt % in H₂O) and acrylonitrile (12.4 mL) were added. The resulting mixture was stirred for 3 days at 50° C. The solution was cooled to room temperature and CH₂Cl₂ (30 mL) was added. Solids were removed by filtration. The filtrate was extracted with a 1N aqueous HCl solution (15 mL). The aqueous layer was washed with CH₂Cl₂. The pH of the aqueous layer was adjusted to pH=12-13 with a 2N aqueous NaOH solution, and extracted with CH₂Cl₂. The organic layers were combined and washed with a saturated aqueous NaCl solution, dried over anhydrous MgSO₄, filtered and concentrated in vacuum to obtain Michael addition product (“Compound 2-A”). ¹H NMR (300 MHz, CDCl₃, ppm) 3.70 (t, J=6.03 Hz, 6H), 3.46 (s, 6H), 2.62 (t, J=6.00 Hz, 6H), 1.60 (bs, 2H).

To a round bottom flask was added Compound 2-A (6 g) and methanol (40 mL). The solution was cooled to −78° C. and SOCl₂ was slowly added. The reaction mixture was raised to 0° C. and stirred for 30 minutes, then at room temperature for 30 minutes, and at 60° C. for 3 days. The reaction mixture was cooled to room temperature, and the solids were removed by filtration. Filtrate was concentrated and the resulting residue was dissolved in 30 mL of CH₂Cl₂ and extracted with 30 mL of H₂O. The aqueous layer was washed with CH₂Cl₂ and the pH was adjusted to pH=12-13 using aqueous NaOH solution. The aqueous solution was extracted with 20 mL of CH₂Cl₂. The organic layers were combined, washed with a saturated aqueous NaCl solution, dried over MgSO₄, filtered and concentrated to yield the esterified compound (“Compound 2-B”). ¹H NMR (300 MHz, CDCl₃, ppm) 3.71 (t, J=6.03 Hz, 6H+s, 9H), 3.32 (s, 6H), 2.58 (t, J=6.31 Hz, 6H), 1.82 (bs, 2H).

To a round bottom flask was added Compound 2-B (2 g), 4-pentynoic acid (0.398 g), 4-hydroxybenzotriazole monohydrate (HOBt.H₂O) and CH₂Cl₂ (20 mL). To the resulting solution was added Et₃N (1.41 mL) at room temperature, EDCI.HCl (1.010 g). The reaction solution was stirred for 12 hrs at 40° C. The reaction mixture was cooled to room temperature and washed with 10 mL of a 0.5N aqueous HCl solution. The organic layer was separated and washed with 20 mL of a saturated aqueous NaCl solution, dried over MgSO₄, filtered, concentrated and purified to yield dimethyl 3,3′-((2-((3-methoxy-3-oxopropoxy)methyl)-2-(pent-4-ynamido)propane-1,3-diyl)bis(o xy))dipropionate (“Compound B”). ¹H NMR (300 MHz, CDCl₃, ppm) 6.12 (bs, 1H), 3.71 (t, J=6.03 Hz, 6H+s, 15H), 2.55 (t, J=6.20 Hz, 6H), 2.51-2.49 (m, 2H), 2.43-2.38 (m, 2H), 1.97 (t, J=6.31 Hz, 1H).

### Example 3

### Synthesis of Linker 3,3′-((2-((2-carboxyethoxy)methyl)-2-(3-(1-(4-oxobutyl)-1H-1,2,3-triazol-5-yl)propanamido)propane-1,3-diyl)bis(oxy))dipropionic acid

To a round bottom flask was added Compound A (0.366 g, Example 1), Compound B (1.07 g, Example 2) and THF (10 mL). The mixture was stirred at room temperature until a homogeneous solution was obtained after which sodium ascorbate (0.138 g) and 10 mL H₂O solution of copper (II) sulfate pentahydrate (CuSO₄5H₂O) were added at room temperature. The resulting solution was stirred for 4 hours at room temperature. The solvent was removed and the residue was diluted with 10 mL of H₂O and extracted with CH₂Cl₂. The organic layers were combined, washed a saturated aqueous NaCl solution, dried over MgSO₄, filtered, concentrated, and purified to produce dimethyl 3,3′-((2-(3-(1-(3-(1,3-dioxolan-2-yl)propyl)-1H-1,2,3-triazol-5-yl)propanamido)-2-((3-methoxy-3-oxopropoxy)methyl)propane-1,3-diyl)bis(oxy))dipropionate (“Compound 3-1”). ¹H NMR (300 MHz, CDCl₃, ppm) 7.39 (s, 1H), 6.04 (s, 1H), 4.88 (t, J=4.38 Hz, 1H), 4.37 (t, J=7.28 Hz, 2H), 3.97-3.94 (m, 2H), 3.87-3.85 (m, 2H), 3.70-3.66 (m, 21H), 2.99 (t, J=7.28 Hz, 2H), 2.59-2.52 (m, 8H), 2.06-2.00 (m, 2H), 1.72-1.67 (m, 2H); ¹³C NMR (75 MHz, CDCl₃, ppm): 172.1, 146.9, 121.3, 103.6, 69.2, 66.7, 65.0, 59.7, 51.7, 49.9, 36.3, 34.7, 30.4, 24.7, 21.5.

To a round bottom flask was added Compound 3-1 (1.230 g), MeOH (12 mL) and an aqueous KOH solution (2.0 M, 2.993 mL). The mixture was stirred for 2 hours at room temperature. Concentration of the mixture produced potassium 3,3′-((2-(3-(1-(3-(1,3-dioxolan-2-yl)propyl)-1H-1,2,3-triazol-5-yl)propanamido)-2-((2-carboxylatoethoxy)methyl)propane-1,3-diyl)bis(oxy))dipropionate (“Compound 3-2”). ¹H NMR (300 MHz, DMSO-d₆, ppm) 8.05 (s, 2H), 4.80 (t, J=4.50 Hz, 1H), 4.32 (t, J=6.80 Hz, 2H), 3.89-3.85 (m, 2H), 3.84-3.80 (m, 2H), 3.54-3.50 (m, 12H), 2.77 (t, J=7.00 Hz, 2H), 2.47-2.43 (m, 2H), 2.09-2.03 (m, 6H), 1.91-1.81 (m, 2H), 1.20-1.16 (m, 2H); ¹³C NMR (75MHz, DMSO-d₆, ppm): 174.5, 172.1, 146.8, 122.8, 103.5, 70.0, 69.1, 64.7, 60.2, 49.4, 35.4, 30.6, 25.0, 22.0.

To a round bottom flask was added Compound 3-2 (1.00 g), MeOH (10 mL), and an aqueous HCl solution (1.0 M, 4.356 mL). The mixture was stirred for 2 hours at room temperature after which methanol and water were removed, filtered, and the solid residue was washed with 40 mL of a co-solvent (CH₂Cl₂/MeOH=10/1). The filtrate was concentrated in a rotary evaporator, and then dried to produce 3,3′-((2-((2-carboxyethoxy)methyl)-2-(3-(1-(4-oxobutyl)-1H-1,2,3-triazol-5-yl)propanamido)propane-1,3-diyl)bis(oxy))dipropionic acid (“Compound 3-3”). ¹H NMR (300 MHz, DMSO-d₆, ppm) 7.77 (s, 1H), 7.13 (s, 1H), 4.79 (t, J=4.46 Hz, 1H), 4.32 (t, J=6.89 Hz, 2H), 3.89-3.81 (m, 2H), 3.77-3.72 (m, 2H), 3.54-3.50 (m, 12H), 2.78 (t, J=6.00 Hz, 2H), 2.57-2.52 (m, 8H), 1.90-1.81 (m, 2H), 1.56-1.50 (m, 2H); ¹³C NMR (75MHz, DMSO-d₆, ppm): 173.3, 171.9, 146.6, 122.3, 103.4, 68.5, 67.0, 64.7, 60.3, 49.4, 35.8, 35.2, 30.6, 25.0, 21.8.

To a round bottom flask was added Compound 3-3 (0.4735 g), PPTS (pyridinium p-toluenesulfonate, 41.4 mg) and 4:1 mixture of acetone/H₂O (5 mL). The reaction mixture was refluxed for 24 hours, and then cooled to room temperature. The resulting solution was concentrated and dried to produce 3,3′-((2-((2-carboxyethoxy)methyl)-2-(3-(1-(4-oxobutyl)-1H-1,2,3-triazol-5-yl)propanamido)propane-1,3-diyl)bis(oxy))dipropionic acid as a desired linker. ¹H NMR (300 MHz, DMSO-d₆, ppm) 12.16 (broad s, 3H), 9.63 (t, J=0.99 Hz, 1H), 7.78 (s, 1H), 7.10 (s, 1H), 4.31 (t, J=7.00 Hz, 2H), 3.59-3.55 (m, 12H), 2.79 (t, J=7.14 Hz, 2H), 2.52-2.46 (m, 10H), 2.08-2.05 (m, 2H).

### Example 4

### Synthesis of Other Linkers

Using similar procedures as those described in Examples 1-3 above, the following linkers were also prepared: 3,3′-((2-((2-carboxyethoxy)methyl)-2-(3-(1-(2-(2-(2-oxoethoxy)ethoxy)ethyl)-1H-1,2,3-triazol-5-yl)propanamido)propane-1,3-diyl)bis(oxy))dipropionic acid; 3,3′-((2-((2-carboxyethoxy)methyl)-2-(3-(1-(2-(2-(2-(oxiran-2-ylmethoxy)ethoxy)ethoxy)ethyl)-1H-1,2,3-triazol-5-yl)propanamido)propane-1,3-diyl)bis(oxy))dipropionic acid; 3,3′-((2-((2-carboxyethoxy)methyl)-2-(3-(1-(3-(oxiran-2-yl)propyl)-1H-1,2,3-triazol-5-yl)propanamido)propane-1,3-diyl)bis(oxy))dipropionic acid.

### Example 5

### Attaching a Linker to RCM

About 2 to 5 g of a linker produced according to the above Examples was dissolved in N,N-dimethylformamide (400 mL). A piece of RCM was soaked in the solution, and the wet RCM is heated at 100° C. in vacuum until dryness. The resulting RCM is washed with ethyl alcohol, and dried in vacuum to produce an RCM having the linker attached to its surface.

### Example 6

### Attaching a Linker to RCM

A linker is dissolved in a mixture of N,N-dimethylformamide (40 mL) and dichloromethane (160 mL). To this solution is added N,N′-dicyclohexylcarbodiimide (0.31 g) and 4-(dimethylamino)pyridine (0.18 g). When all the solids is dissolved, a piece of RCM is soaked in the solution for 2 hours at room temperature. The RCM is then removed, washed with copious ethyl alcohol, and dried in vacuum to produce an RCM having the linker attached to its surface.

### Example 7

### One-Step Quantitative Detection of Acute Myocardial Infarction Biomarker cTnI (Cardiac Troponin I) Target Molecule Using Lateral Flow Type Rapid Test Kit Including the RCM Matrix

This example illustrates a one-step quantitative test of a cTnI biomarker in the human blood using an RCM having a covalently attached capture antibody (i.e., a receptor molecule), and allowing the complex formed from cardiac troponin I (cTnI) and the detection (i.e., a signal generation) molecule tagged with a fluorescent dye to interact with the capture antibody in a lateral flow type diagnostic kit.

cTnI-specific capture antibody (2.0 mg/ml, 0.35 ml/cm dispensing rate, twice repeated dispensing, 0.56 mg/4 mm strip, 4T21 clone 560, HyTest, Finland) was dispensed on the RCM that was produced using the procedure described in Example 5 or 6 above. For comparison cTnI-specific capture antibody (2.0 mg/ml, 0.35 ml/cm dispensing rate, twice repeated dispensing, 0.56 mg/4 mm strip, 4T21 clone 560, HyTest, Finland) was dispensed on nitrocellulose (“NCM”) membrane, which binds cTnI-specific antibody noncovalently.

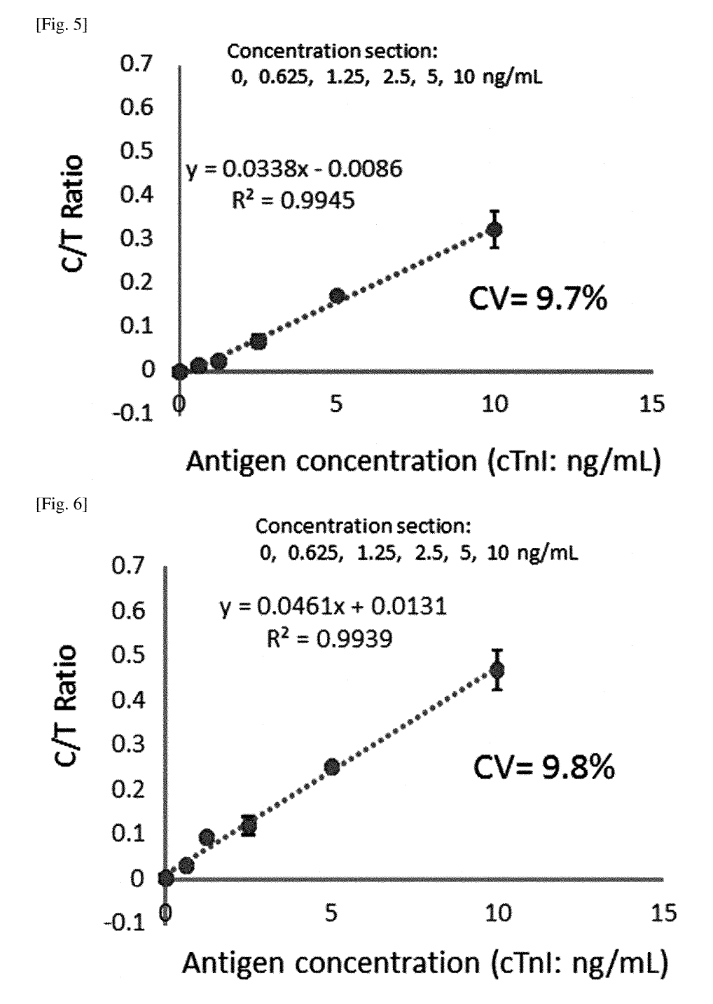

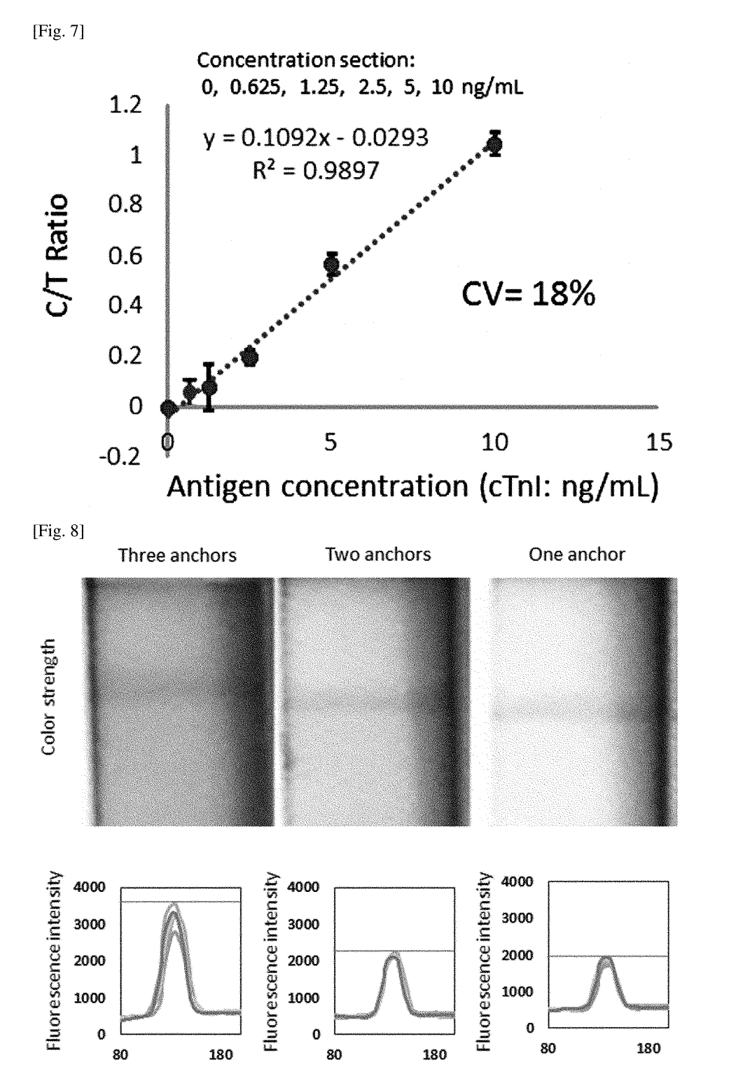

cTnI antigen (8T62, HyTest, Finland) was diluted with human serum (Sigma-Aldrich, Louis, Mo.) to produce concentrations of 0, 0.625, 1.25, 2.5, 5, and 10 ng/mL. cTnI antigen was then attached to a detection antibody, i.e., a signal generating molecule, (5.0 mg/ml, 20 ml/test, 4T21 clone 19C7, HyTest, Finland) which was tagged with fluorescent dyes (i.e., the signal generating moiety) (FLM647s, BioActs, Korea; excitation at 650 nm and emission at 668 nm). The resulting complex was allowed to flow laterally on the RCM having cTnI-specific capture antibody. The fluorescent signal was measured by using a fluorescence reader. From the measured fluorescent intensity profiles, areas of a control line region (“C”) and a test line region (“T”) were integrated, and a C/T ratio was calculated to generate a graph (the C/T ratio vs. a target molecule (cTnI) concentration). The result are illustrated in FIGS. 5-7.

Comparison of a determination coefficient R² and a coefficient of variation CV showed the RCM that was attached to a linker having an aldehyde functional group as the RCM linking functional group had R²=0.9945 and CV=9.7%, the RCM produced using a linker having an epoxide functional group as the RCM linking functional group had R²=0.9939 and CV=9.8%, in contrast the NCM had R²=0.9897 and CV=18.6% (see FIGS. 5, 6, and 7). The results clearly show that in a lateral flow type test, the quantitative analysis method using the RCM of the present invention is significantly more reproducible and reliable than the quantitative analysis method using an NCM.

### Example 8

### Different Numbers of Anchoring Group (the Numbers of Branch Moiety) of the Linker

This example illustrates results of linkers with different number of RCM linking functional groups.

Using the procedure of Example 7, RCMs were prepared using linkers having one, two, or three carboxylic acid groups as the RCM linking functional groups. cTn-specific capture antibody was covalently attached to these three different RCMs (FIG. 8).

cTnI antigen (8T62, HyTest, Finland) was diluted with human serum (Sigma-Aldrich, Louis, Mo.) to make a solution of 10 ng/mL, and mixed with a detection antibody, i.e., signal generating molecule, (5.0 mg/ml, 20 ml/test, 4T21 clone 19C7, HyTest, Finland) which was tagged with fluorescent dyes, i.e., signal generating moiety, (FLM647s, BioActs, Korea, excitation at 650 nm and emission at 668 nm). Alternatively, a detection antibody (1.0 ml/test, 4T21 clone 19C7, HyTest, Finland) was conjugated with colloidal gold particles, i.e., a signal generating moiety, having a size of 40 nm at a concentration of 20 O.D. The fluorescence signal was measured by using a fluorescence reader. The visual absorption arising from the colloid gold particles was verified with the naked eyes. Fluorescent intensity values measured using the fluorescence reader were plotted. (see FIG. 9). As the results show the linker with three anchoring groups (i.e., RCM linking functional groups) showed stronger signal than those for two anchoring groups or one anchoring group (see FIG. 9). Similarly, the visual absorption was the strongest for the linker having three anchoring groups.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. A regenerated cellulose membrane (RCM) configured for use in analysis of a fluid sample for the presence of a target molecule, said regenerated cellulose membrane comprising:
a regenerated cellulose membrane (RCM) having a plurality of RCM functional groups on its surface;
a linker having a distal end and a proximal end, wherein said proximal end comprises at least one RCM linking functional group such that said RCM linking functional group is attached to said RCM functional group, and wherein said distal end of said linker comprises a receptor linking functional group that is attached to a functional group of a receptor molecule, wherein said receptor molecule is adapted for binding to a target molecule when said target molecule is present in said fluid sample.

2. The regenerated cellulose membrane according to claim 1, wherein said linker comprises a plurality of said RCM linking functional groups such that said plurality of said RCM linking functional groups in said linker is attached to said plurality of RCM functional groups.

3. The regenerated cellulose membrane according to claim 1, wherein said receptor molecule comprises an enzyme, a ligand, an antigen, an antibody, a DNA or a fragment thereof, an RNA or a fragment thereof, a PNA or a fragment thereof, an aptamer, a ligand, a lipid, a hormone, an inorganic molecule, a cell, a peptide, or a small organic molecule.

4. The regenerated cellulose membrane according to claim 1, wherein said RCM functional group is selected from the group consisting of a hydroxyl group, an amino group, a thiol group, a carboxylic acid, aldehyde, a phosphonate group, a formyl group, a carbonyl group, a carboxylate group, an ether group, a nitro group, a sulfonic acid group, a phosphine group, and an epoxide group.

5. The regenerated cellulose membrane according to claim 1, wherein said linker is a moiety of the formula: 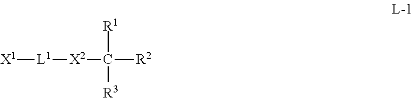
wherein
X¹ is said receptor linking function group;
X² is a branch linking functional group;
L¹ is a first linker moiety having at least four atoms in a chain;
R¹ is a moiety of the formula -[L²-Q²]ₐ-{(L³-Q³)_{b}-[(L⁴-Q⁴)_{c}-(L⁵-Y)ₓ]_{y}}_{z};
each of R² and R³ is independently hydrogen, alkyl, or a moiety of the formula:
-[L²-Q²]ₐ-{(L³-Q³)_{b}-[(L⁴-Q⁴)_{c}-(L⁵-Y)ₓ]_{y}}_{z}
each of a, b, and c is independently 0 or 1;
x is 1 when c is 0 or when c is 1, x is an integer from 1 to the oxidation state of Q⁴-1;
y is 1 when b is 0 or when b is 1, y is an integer from 1 to the oxidation state of Q³-1;
z is 1 when a is 0 or when a is 1, z is an integer from 1 to the oxidation state of Q²-1;
each of Q², Q³ and Q⁴ is independently a branch atom having the oxidation state of at least 3; and
each of L², L³, L⁴, and L⁵ is independently a linker moiety having at least four atoms in a chain;
provided the product of x, y, and z is at least 1.

6. The regenerated cellulose membrane according to claim 5, wherein X¹ is selected from the group consisting of aldehyde, epoxide, halide, mesylate, tosylate, carboxylate, thioester, sulfonate, and phosphonate.

7. The regenerated cellulose membrane according to claim 5, wherein L¹ comprises at least nine chain atoms.

8. The regenerated cellulose membrane according to claim 7, wherein L¹ comprises a heteroaryl within said chain atoms.

9. The regenerated cellulose membrane according to claim 5, wherein X² is selected from the group consisting of an ester, an amide, a phosphonate and sulfonate.

10. The regenerated cellulose membrane according to claim 5, wherein at least one of R² and R³ is a moiety of the formula: -[L²-Q²]ₐ-{(L³-Q³)_{b}-[(L⁴-Q⁴)_{c}-(L⁵-Y)ₓ]_{y}}_{z}, wherein L², L³, L⁴, L⁵, Q², Q³, Q⁴, Y, a, b, c, x, y and z are as defined in claim 5.

11. The regenerated cellulose membrane according to claim 5, wherein Y selected from the group consisting of aldehyde, epoxide, halide, carboxylate, thioester, phosphonate, sulfonate, mesylate and tosylate.

12. The regenerated cellulose membrane according to claim 1, wherein a plurality of said receptor molecule is spaced in regular intervals on the surface of said RCM.

13-31. (canceled)

32. A compound of the formula: 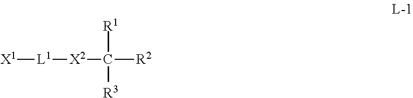
wherein
X¹ is a receptor linking function group;
X² is a branch linking functional group;
L¹ is a first linker moiety having at least four atoms in a chain;
R¹ is a moiety of the formula -[L²-Q²]ₐ-{(L³-Q³)_{b}-[(L⁴-Q⁴)_{c}-(L⁵-Y)ₓ]_{y}}_{z};
each of R² and R³ is independently hydrogen, alkyl, or a moiety of the formula:
-[L²-Q²]ₐ-{(L³-Q³)_{b}-[(L⁴-Q⁴)_{c}-(L⁵-Y)ₓ]_{y}}_{z}
each of a, b, and c is independently 0 or 1;
xis 1 when c is 0 or when c is 1, x is an integer from 1 to the oxidation state of Q⁴-1;
y is 1 when b is 0 or when b is 1, y is an integer from 1 to the oxidation state of Q³-1;
z is 1 when a is 0 or when a is 1, z is an integer from 1 to the oxidation state of Q2-1;
each of Q², Q³ and Q⁴ is independently a branch atom having the oxidation state of at least 3; and
each of L², L³, L⁴, and L⁵ is independently a linker moiety having at least four atoms in a chain;
provided the product of x, y, and z is at least 1.

33. The compound according to claim 32, wherein X¹ is selected from the group consisting of aldehyde, epoxide, halide, mesylate, tosylate, carboxylate, thioester, sulfonate, and phosphonate.

34. The compound according to claim 32, wherein L¹ comprises at least nine chain atoms.

35. The compound according to claim 34, wherein L¹ comprises a heteroaryl within said chain atoms.

36. The compound according to claim 32, wherein X² is selected from the group consisting of an ester, an amide, a phosphonate and sulfonate.

37. The compound according to claim 32, wherein at least one of R² and R³ is a moiety of the formula: -[L²-Q²]ₐ-{(L³-Q³)_{b}-[(L⁴-Q⁴)_{c}-(L⁵-Y)ₓ]_{y}}_{z}, wherein L², L³, L⁴, L⁵, Q², Q³, Q⁴, Y, a, b, c, x, y and z are as defined in claim 32.

38. The compound according to claim 32, wherein Y selected from the group consisting of aldehyde, epoxide, halide, carboxylate, thioester, phosphonate, sulfonate, mesylate and tosylate.

39-50. (canceled)
